(19) United States
(12) Patent Application Publication
(10) Pub. No.: US 2026/0096825 A1
(43) Pub. Date: Apr. 9, 2026

(54) ULTRASONIC INTRAVASCULAR STENT DEVICE

(71) Applicant: Zhijun PENG, Foshan (CN)

(72) Inventor: Zhijun PENG, Foshan (CN)

(21) Appl. No.: 18/988,856

(22) Filed: Dec. 19, 2024

(30) Foreign Application Priority Data
Oct. 6, 2024 (CN) 2024113876830

(51) Int. Cl. A61B 17/22; A61B 17/00; A61F 2/90

(52) U.S. Cl. CPC A61B 17/22012; A61F 2/90; A61B 2017/00221; A61B 2017/00345; A61B 2017/00734; A61F 2250/0093

(57) ABSTRACT

An ultrasonic intravascular stent device, including a stent and a micro ultrasonic vibration module mounted on the stent. The micro ultrasonic vibration module having has a housing and a housing cover, an accommodating cavity defined therebetween, and an ultrasonic vibration assembly and a driving control assembly provided in the accommodating cavity.

### BACKGROUND OF THE INVENTION

The present invention relates to the field of heart stents, and in particular to an ultrasonic intravascular stent device.

An intravascular stent is a medical instrument commonly used in interventional operations of heart, cerebral vessels, renal arteries, aorta, and the like, and has a good effect of unclogging arterial vessels. The use of intravascular stent refers to the implantation of a stent expanded and opened up at the site of health disorder by expansion of a balloon, so as to achieve the purposes of supporting a narrowed or a clogged blood vessel, reducing contraction and restenosis of the blood vessel, and maintaining an unobstructed blood flow in the blood vessel. At present, stents used in different body parts mainly include a coronary stent, a cerebrovascular stent, a renal artery stent, an aortic stent, and the like. The main material of a stent nowadays is stainless steel, nickel-titanium alloy, or cobalt-chromium alloy. From its earliest appearance in the 1980s up till now, stents of different types like metal stents, drug-coated stents, and bioabsorbable stents have been developed. However, at present, all kinds of intravascular stents of various shapes and materials that are implanted into human blood vessels only play a role in preventing the contraction and restenosis of the blood vessels, but cannot assist in self-repair of the inner walls of the blood vessels or ablate the thrombus in the blood vessels. Moreover, since pathological changes of some patients are complicated, including calcification, bifurcation, and tortuosity, coronary artery thrombosis and vascular dissection may occur during heart stent surgeries, resulting in a potential risk of vascular occlusion, which is common in clinical practice. Therefore, the current intravascular stent products are still significantly defective, and need to be improved and optimized to better serve the patients.

### BRIEF SUMMARY OF THE INVENTION

The present invention intends to solve the above problems and disadvantages by providing an ultrasonic intravascular stent device. According to the present invention, by mounting a micro ultrasonic module on the stent and utilizing an ultrasonic cavitation effect generated by the ultrasonic waves, the intravascular stent device can promote blood circulation, relieve soft tissues, reduce swelling, eliminate inflammation, improve the nutritional status of tissues, ablate a thrombus, and prevent the occurrence of thrombosis, thereby achieving an effect of assisting in self-repair of inner walls of blood vessels.

The present invention is achieved as follows:

An ultrasonic intravascular stent device, comprising a stent, wherein a micro ultrasonic vibration module is mounted on the stent; the micro ultrasonic vibration module comprises a housing and a housing cover assembled to each other, wherein an accommodating cavity is defined between an enclosure formed by the housing and the housing cover; the micro ultrasonic vibration module also comprises an ultrasonic vibration assembly and a driving control assembly, both disposed in the accommodating cavity; the ultrasonic vibration assembly comprises a slidable block, resilient tabs disposed at two ends of the slidable block respectively, and at least one driving coil; at least one permanent magnet block is provided on the slidable block; the resilient tabs provides buffering and bouncing functions against the slidable block as the slidable block slides; said at least one driving coil cooperates with said at least one permanent magnet block to generate a magnetic driving effect; the driving control assembly comprises a circuit board on which a wireless communication module is disposed, and a battery and a wireless charging coil which are electrically connected to the circuit board; said at least one driving coil is electrically connected to the circuit board to drive the ultrasonic vibration assembly to work.

Preferably, the accommodating cavity is provided with a partition plate that partitions the accommodating cavity into a first working chamber and a second working chamber; the ultrasonic vibration assembly is disposed in the first working chamber, and the driving control assembly is disposed in the second working chamber.

Preferably, the second working chamber is stacked on the first working chamber.

Preferably, said at least one driving coil is disposed on the partition plate, and the slidable block is disposed on the housing cover.

Preferably, sensors electrically connected to the circuit board are further disposed at two ends of the housing respectively.

Preferably, transitional inclined planes are further provided on peripheral sides of the housing.

The present invention has the following beneficial effects: by mounting a micro ultrasonic module in the stent and utilizing an ultrasonic cavitation effect generated by the ultrasonic waves, the intravascular stent device can promote blood circulation, relieve soft tissues, reduce swelling, eliminate inflammation, improve the nutritional status of tissues, ablate a thrombus, and prevent the occurrence of thrombosis, thereby achieving an effect of assisting in self-repair of inner walls of blood vessels. Also, drugs intake of the user can be greatly reduced due to the use of the present invention, and hence side effects of the drugs harmful to the body are greatly reduced, and the user's life may therefore by prolonged.

### BRIEF DESCRIPTION OF THE DRAWINGS

FIG. 1 is a schematic overall structural view of the present invention;

FIG. 2 is a schematic structural bottom plan view of the present invention;

FIG. 3 is a schematic perspective view of a micro ultrasonic vibration module of the present invention;

FIG. 4 is a first exploded structural view of a micro ultrasonic vibration module of the present invention;

FIG. 5 is a second exploded structural view of a micro ultrasonic vibration module of the present invention, where the housing cover is omitted from illustration;

FIG. 6 is a schematic cross-sectional view of a housing, a partition plate, and a housing cover of the present invention; and

FIG. 7 is a schematic diagram of a use state of the present invention when charging.

### DETAILED DESCRIPTION OF THE INVENTION

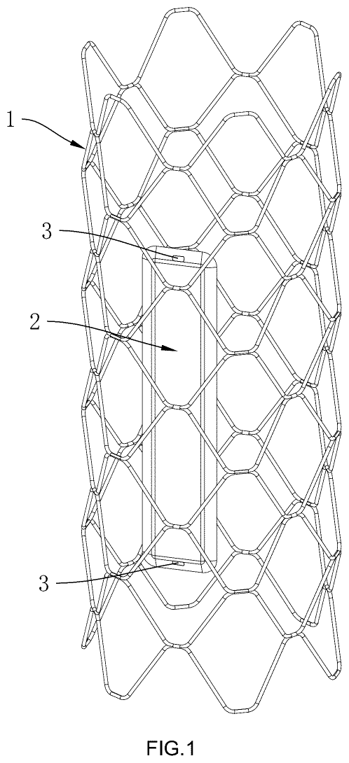

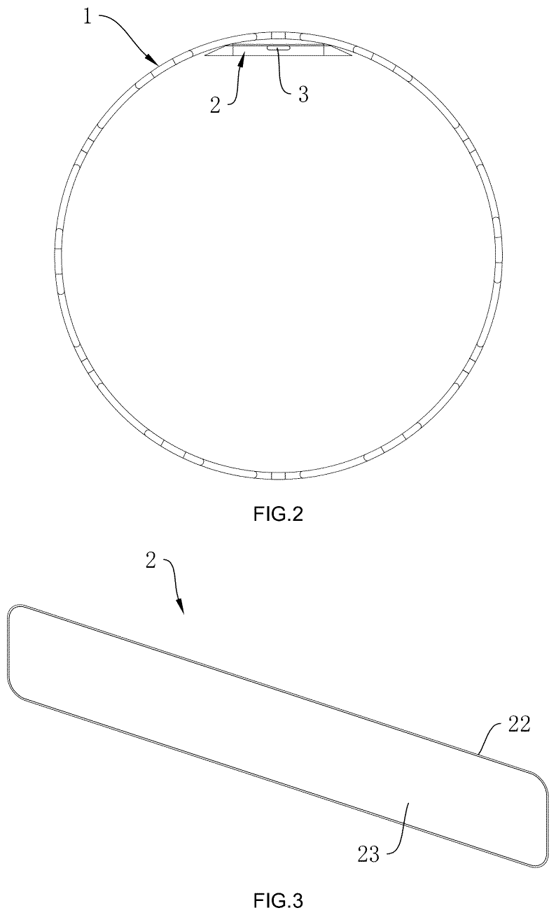

As shown in FIG. 1 and FIG. 2, the ultrasonic intravascular stent device of the present invention comprises a stent 1. In order to achieve the purposes of the present invention, a micro ultrasonic vibration module 2 is mounted on the stent 1. As shown in FIG. 3 to FIG. 5, the micro ultrasonic vibration module 2 comprises a housing 22 and a housing cover 23 assembled to each other, wherein an accommodating cavity 21 is defined between an enclosure formed by the housing 22 and the housing cover 23; the micro ultrasonic vibration module 2 also comprises an ultrasonic vibration assembly 24 and a driving control assembly 25, both disposed in the accommodating cavity 21. The ultrasonic vibration assembly 24 comprises a slidable block 242, resilient tabs 243 disposed at two ends of the slidable block 242 respectively, and at least one driving coil 244; at least one permanent magnet block 241 is provided on the slidable block 242; the resilient tabs 243 provides buffering and bouncing functions against the slidable block 242 as the slidable block 242 slides; said at least one driving coil 244 cooperates with said at least one permanent magnet block 241 to generate a magnetic driving effect. The driving control assembly 25 comprises a circuit board 252 on which a wireless communication module 251 is disposed, and a battery 253 and a wireless charging coil 254 which are electrically connected to the circuit board 252. Said at least one driving coil 244 is electrically connected to the circuit board 252 to drive the ultrasonic vibration assembly 24 to work.

The working principle of the ultrasonic vibration assembly 24 is as follows: The battery 253 supplies power to said at least one driving coil 244 through the circuit board 252, and a current flows into said at least one driving coil 244 to generate a magnetic field, which drives said at least one permanent magnet block 241 and thus the slidable block 242 to reciprocate, thereby generating high-frequency vibrations, which propagate through a medium such as gas, liquid, and solid to form ultrasonic waves.

Compared with conventional eccentric motor, the ultrasonic vibration assembly 24 has the advantage of reducing, if not eliminating, motion inertia, and can therefore realize rapid start/stop of the reciprocation of the slidable block 242; further, the ultrasonic vibration assembly 24 does not require any space for eccentric rotation as in the case of using eccentric motor in the prior art, as a result, an overall thickness of the micro ultrasonic vibration module 2 can be smaller. Apart from using the aforementioned ultrasonic vibration assembly 24 as described above, the present invention can be alternatively implemented by using an ultrasonic transducer or an ultrasonic vibration motor in lieu of the ultrasonic vibration assembly 24. However, product performance is slightly poorer if the ultrasonic vibration motor is used.

In practical application, said at least one driving coil 244, the wireless charging coil 254, and the battery 253 are all electrically connected to the circuit board 252 through conducting wires, and the wireless communication module 251 is built-in to the circuit board 252. Moreover, the wireless communication module 251 is an ultra-micro Bluetooth® communication module, WiFi® communication module, or the like. By utilizing a wireless communication module 251 to be linked to a device like a computer, a smartphone, or a tablet computer, remote operations, monitoring, and data transmissions of the micro ultrasonic vibration module 2 are realized.

Specifically, the accommodating cavity 21 may be a recess on the housing 22, or may be a recess on the housing cover 23.

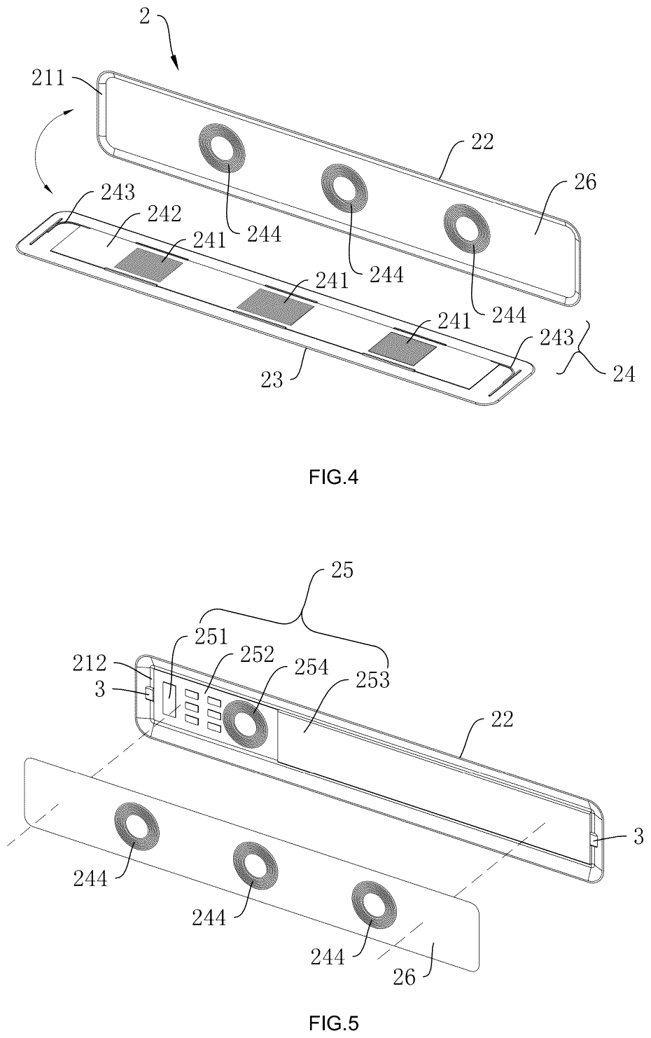

As shown in FIG. 4 to FIG. 6, the accommodating cavity 21 is provided with a partition plate 26 for partitioning the accommodating cavity 21 into a first working chamber 211 and a second working chamber 212. The ultrasonic vibration assembly 24 is disposed in the first working chamber 211, and the driving control assembly 25 is disposed in the second working chamber 212. By partitioning the accommodating cavity 21 into two independent chambers, the ultrasonic vibration assembly 24 and the driving control assembly 25 can be prevented from interfering with each other, so that the operations of the two are more stable and reliable.

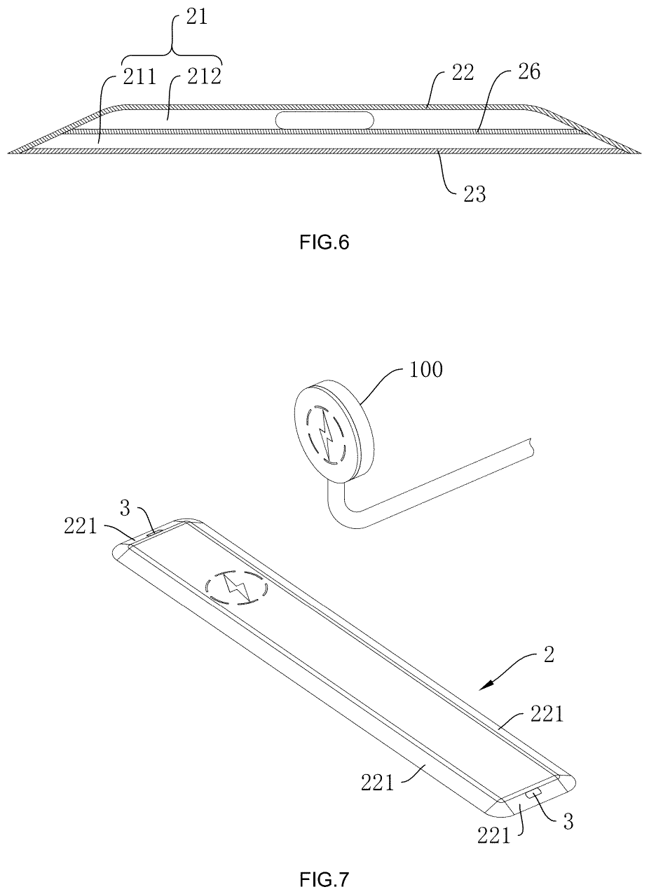

As shown in FIG. 6, the second working chamber 212 is stacked on the first working chamber 211. This enables the ultrasonic vibration assembly 24 to be disposed along a lengthwise direction of the micro ultrasonic vibration module 2, so that the vibration frequencies of various parts of the micro ultrasonic vibration module 2 are more balanced, and so the ultrasonic vibration treatment effect is better. Moreover, by positioning the second working chamber 212 above the first working chamber 211, the wireless charging coil 254 is positioned closer to a top surface of the housing 22, so that the wireless charging coil 254, as a receiving end of an external wireless charging device, can be closer to a transmitting end 100 of the external wireless charging device during wireless charging, thereby enabling the wireless transmission of electric energy to be more stable, reliable, and difficult to interrupt.

As shown in FIG. 4, said at least one driving coil 244 is disposed on the partition plate 26, and the slidable block 242 is disposed on the housing cover 23. In this way, it is unnecessary to move the conducting wires out onto the housing cover 23, thereby facilitating assembly and preventing the conducting wires from affecting the sliding of the slidable block 242.

As shown in FIGS. 5 and 7, sensors 3 electrically connected to the circuit board 252 are further disposed at two ends of the housing 22 respectively. Each of the sensors 3 can be a heart rate detection sensor, a blood oxygen detection sensor, a body temperature detection sensor, an image sensor mounted with a micro camera, or the like. In this way, it is convenient to observe and detect the health condition of the user, thereby increasing the usage functions of the ultrasonic intravascular stent device.

In practical application, the housing 22 and the housing cover 23 are made of a same material as the stent 1, such as stainless steel, nickel-titanium alloy, or cobalt-chromium alloy.

In practice, as shown in FIG. 2, the micro ultrasonic module 2 is disposed on an inner wall of the stent 1. In this way, the fixation is simple, the deployment of the stent 1 is not hindered, and vision of the micro camera is not blocked. More specifically, the micro ultrasonic module 2 can be fixed on the inner wall of the intravascular stent 1 by welding. Alternatively, a retaining ring is provided on the inner wall of the intravascular stent 1, and a hook is provided on the micro ultrasonic module 2, such that the micro ultrasonic module 2 is fixed to the inner wall of the intravascular stent 1 by hooking the hook onto the retaining ring.

As shown in FIG. 7, transitional inclined planes 221 are further provided on peripheral sides of the housing 22. In this way, when blood passes by, the blood can smoothly flow passed the housing 22 without being blocked by the housing 22, thereby ensuring normal circulation of the blood.

What is claimed is:

1. An ultrasonic intravascular stent device, comprising a stent, wherein a micro ultrasonic vibration module is mounted on the stent;
the micro ultrasonic vibration module comprises a housing and a housing cover assembled to each other, wherein an accommodating cavity is defined between an enclosure formed by the housing and the housing cover; the micro ultrasonic vibration module also comprises an ultrasonic vibration assembly and a driving control assembly, both disposed in the accommodating cavity;
the ultrasonic vibration assembly comprises a slidable block, resilient tabs disposed at two ends of the slidable block respectively, and at least one driving coil; at least one permanent magnet block is provided on the slidable block; the resilient tabs provides buffering and bouncing functions against the slidable block as the slidable block slides; said at least one driving coil cooperates with said at least one permanent magnet block to generate a magnetic driving effect;
the driving control assembly comprises a circuit board on which a wireless communication module is disposed, and a battery and a wireless charging coil which are electrically connected to the circuit board; said at least one driving coil is electrically connected to the circuit board to drive the ultrasonic vibration assembly to work.

2. The ultrasonic intravascular stent device of claim 1, wherein the accommodating cavity is provided with a partition plate that partitions the accommodating cavity into a first working chamber and a second working chamber; the ultrasonic vibration assembly is disposed in the first working chamber, and the driving control assembly is disposed in the second working chamber.

3. The ultrasonic intravascular stent device of claim 2, wherein the second working chamber is stacked on the first working chamber.

4. The ultrasonic intravascular stent device of claim 3, wherein said at least one driving coil is disposed on the partition plate, and the slidable block is disposed on the housing cover.

5. The ultrasonic intravascular stent device of claim 1, wherein sensors electrically connected to the circuit board are further disposed at two ends of the housing respectively.

6. The ultrasonic intravascular stent device of claim 1, wherein transitional inclined planes are further provided on peripheral sides of the housing.
